SEQUENCE LISTING (1 sequence)

SEQ ID NO: 1 - DNA, 780 bp, Nandan-4
atacagcggccgcgagctcgggcccccacacgtgtggtctagagctagcctaggctcgagaagcttgtcgacgaattcagattagtcgtcccccccttaactcttaaaatttattttcacccttctgattttaattcttggcttcgtcattggattcaacttacattttgtatactaagaacaattattatcattaattttggagatgactgacatcccatatgaagaagataatttatgtctcctatactaataataagtatgtcatatattgaaaggaaaaactaattattaattgtattgtttggtggaagaactcttaaattaaatatgcttttttaaaagaaattttaggatggatgatcacttgggaagtgttctattttatttacaaaataattttgggtccaattgatgtcctatatagggagaaaaatattgtctatatttttttgatacggttcctatactctatgtcattttgtttggaaaagaattattatctaatattagagagataatgtacgactcttaaattaaagataattatcatccacaaaaaaagagacgatttactaaaaatcatttcctttagaataaggtacgctccttattggggacgactaatcacgaattctggatccgatacgtaacgcgtctgcagcatgcgtggtaccgagctttccctatagtgagtcgtattagagcttggcgtaatcatggtcatagctgtttcctgtgtgaaattgttatccgctcacaattccacacaacatacga

(19) United States
(12) Patent Application Publication
(10) Pub. No.: US 2010/0287820 A1
(43) Pub. Date: Nov. 18, 2010

(54) JATROPHA INTERSPECIFIC HYBRID

(76) Inventors: Koteswara Rao Karanam, Andhra Pradedh (IN); Jayakumar Bhavanasi, Andhra Pradedh (IN)

Correspondence Address: KRAMER & AMADO, P.C., 1725 DUKE STREET, SUITE 240, ALEXANDRIA, VA, 22314, US

(21) Appl. No.: 12/810,759

(22) PCT Filed: Jul. 25, 2008
(86) PCT No.: PCT/IN2008/000474

§ 371 Date: Jun. 25, 2010

(30) Foreign Application Priority Data
Dec. 26, 2007 (IN) 2570/MUM/2007

(51) Int. Cl. A01H 5/00; A01H 1/02; A01H 4/00; C12N 5/00; C10L 1/00

(52) U.S. Cl. 44/307; 800/298; 800/260; 800/268; 800/265; 435/410

(57) ABSTRACT

A novel plant created as an interspecific hybrid of the genus Jatropha designated as Nandan-4 is disclosed. The plant was created by the hybridization of the uncultivated Jatropha gossypifolia with the cultivated Jatropha curcas species. The new interspecific hybrid plant thus invented is found to have unique traits that differed from either parent. This invention relates to the Jatropha interspecific hybrid Nandan-4 plant, the plants generated by any means from a plant part selected from the group consisting of leaves, pollen, embryos, cotyledons, hypocotyls, meristematic cells, roots, root tips, pistils, anthers, flowers, inflorescence, and stems from Nandan-4.

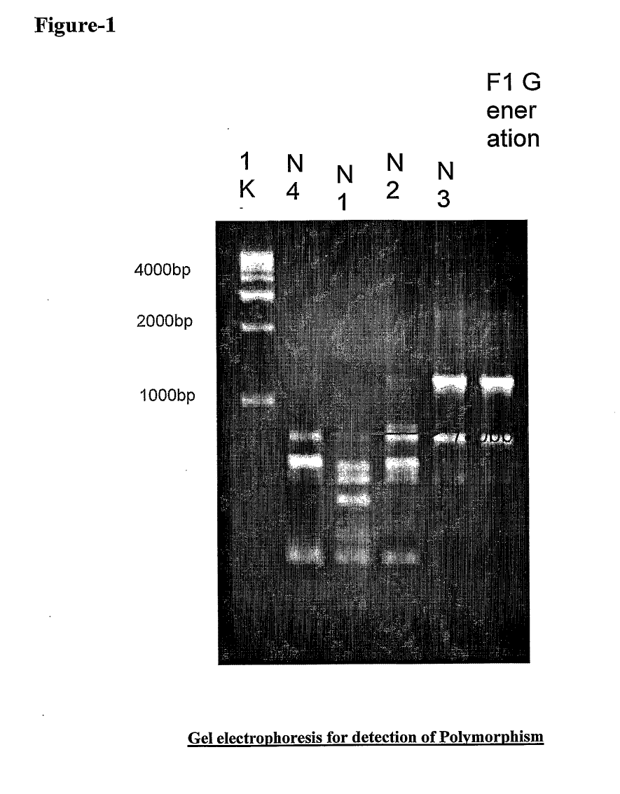

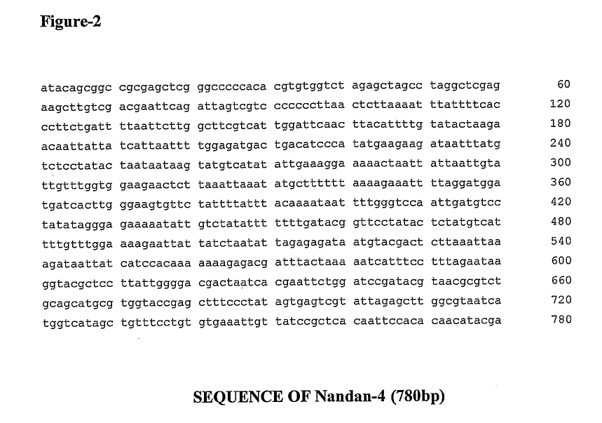

### TECHNICAL FIELD OF THE INVENTION

The present investigation relates to a new and distinctive Jatropha interspecific hybrid designated as Nandan-4. More particularly, it concerns interspecific hybrid plants derived from the cross of Jatropha gossypifolia with Jatropha curcas species with functional gynoecium. All publications cited in this application are herein incorporated by reference.

### BACKGROUND AND PRIOR ART

In India, the tree species considered for biodiesel production are those that are not used as edible oil, which do not complete with food crops for resources and not used in traditional medicine. Among the oil borne tree species, Jatropha curcas is considered to be the most prospective plant due to its hardiness, rapid growth, easy propagation, drought endurance, high oil content, low gestation period and ability to grow on degraded soils and waste lands with low to high rainfall.

Breeding objectives depend on use of the specific crop; increasing yield is a primary objective in all programs. Numerous steps are involved in the development of any novel, desirable cultivar. Hybridization program begins with the analysis and definition of problems and weakness of the current cultivars, followed by the fixation of program goals, and the definition of specific breeding objectives. The next step is selection of parental lines that posse the traits, required to meet the program goals. The goal is to combine in a single cultivar an improved combination, of desirable traits from the parental sources. These important traits may include higher yield, resistance to disease and insect pests, better canopy structure, production of dwarf plants, tolerance to environmental stress, better agronomic characteristics and higher oil content in oil bearing plants.

The goal of Jatropha interspecific hybridization is to develop new, unique and superior interspecific hybrids with higher yield, resistance to disease and insect pests, better canopy structure, production of dwarf plants, tolerance to environmental stress, better agronomic characteristics with synchronous flowering and higher oil content. The breeder initially selects and crosses two species of interest, followed by selection among the many new genetic combinations. The breeder can theoretically generate billions of new and different genetic combinations via crossing. The breeder has no direct control at the cellular level; therefore, two breeders will never develop the same line, or even very similar line, having the similar traits of Jatropha curcas and J. gossypifolia.

As Jatropha curcas is a cross-pollinated crop, the existence of male sterile cultivars would facilitate crossings. Emasculation was not necessary for hybridization in the insect-free greenhouse due to absence of insect vectors and the time lag of anthesis of staminate flowers. The standard routine of bagging should be sufficient in the field. However, to avoid self-pollination if staminate and pistillate flowers were to open simultaneously emasculation could be required as Jatropha curcas is self-compatible and this can be achieved very easily as staminate and pistillate flowers look very distinct.

A similar hybrid was found naturally; refer to the paper titled “Jatropha tanjorensis Ellis & Saroja, a natural interspecific hybrid occurring in Tamil nadu, India”—by A. J. Prabhakaran & M. Sujatha. (Published in Genetic Resources and Crop Evaluation 46: 213-218, 1999. c 1999 Kluwer Academic Publishers. Printed in the Netherlands.), but that does not resemble the present invention, Nandan-4, which has functional gynoecium.

### SUMMARY OF THE INVENTION

The invention provides an interspecific hybrid Jatropha plant or part thereof derived from the cross of Jatropha gossypifolia as female parent and Jatropha curcas as pollen parent. Further provided by the invention are any parts of the interspecific hybrid plant, including a flower, branch cutting, pollen, ovule or any other plant, including a single cell or collection of cells of such a plant. Still further, provided by the invention is an interspecific hybrid Jatropha plant or part thereof clonally propagated from such a plant i.e. any generation of a Jatropha interspecific hybrid plant clonally derived from a plant provided by the present invention. The invention also provides a plant or any part thereof produced by crossing a Jatropha gossypifolia plant to a Jatropha curcas plant, including any clonal propagation thereof.

In addition, the invention provides a process of preparing an interspecific hybrid Jatropha plant or part thereof comprising the steps of, obtaining a cutting of interspecific hybrid Jatropha Nandan-4. Accordingly, any derivatives of a plant derived from the Nandan-4 are specifically within the scope of the invention. As such, those of skill in the art will recognize that once a Jatropha interspecific hybrid plant is prepared from Nandan-4, it may be propagated for a potentially unlimited number of generations. Each plant produced by such a process for the propagation additionally from a part of the present invention

The invention also relates to the hybrid plants and any further progeny or descendants of the hybrid derived by crossing Nandan-4 as a pollen donor. The invention is directed to procedures for producing a Jatropha plant by crossing J. gossypifolia or J. curcas as female parent and Nandan-4 as male parent or vice-versa. The procedures using the interspecific hybrid Jatropha plant Nandan-4 in backcross, hybrid production, crosses to population, clonal propagation, micropropagation and the like are part of this invention. All plants which are a progeny of or descend from Nandan-4 are within the scope of this invention. This is an invention for interspecific Jatropha hybrid Nandan-4 to be used in crosses with other, different, Jatropha plants to produce first filial generation Jatropha hybrid seeds and plants

The present invention also provides regenerable cells for use in tissue culture of Nandan-4. The tissue culture will preferably be capable of regenerating plants having the physiological and morphological characteristics of the foregoing Jatropha interspecific hybrid plant, and of regenerating plants having substantially the same genotype as the foregoing Jatropha curcas plant. Preferably, the regeneration cells in such tissue cultures will be embryos, protoplasts, meristematic cells, callus, pollen, leaves, anthers, pistils, root tips, seeds or stems.

### DEFINITION

In the description and tables which follow, a number of terms are used. In order to provide a clear and consistent understanding of the specification and claims, including the scope to be given such terms, the following definitions are provided:

A plant having essentially all the physiological and morphological characteristics mean a plant having the physiological and morphological characteristics of the cultivar, except for the characteristics derived from the hybridization

Tissue culture: It indicates a composition comprising isolated cells of the same or a different type or a collection of such cells organized into parts of a plant. Exemplary types of tissue cultures are protoplasts, calli, plant meristems, and plant cells that can generate tissue culture that are intact in plants or parts of plants, such as embryos, pollen flowers seeds, inflorescences, leaves, stems, roots, root tips, anthers, and the like.
Regeneration. Regeneration refers to the development of a plant from tissue culture.

Clonal propagation: It is meant any generation of a plant clonally propagated or derived from a plant. Such clonal propagation may be carried out, for example, by taking a cutting of a plant and cultivating the cutting such that it produces roots. The rooted cutting may then be grown as a new plant which is a clone of the starting plant. Clonal propagation may be carried out an essentially unlimited number of times to produce great numbers of clonally propagated plants, each of which is exactly identical to the starting plant from which the cutting is taken.

### DETAILED DESCRIPTION OF THE INVENTION

The invention provides a novel interspecific Jatropha hybrid Nandan-4 plant with unique characteristics like higher internodal length, distantly serrated leaves with pigmented petiole and presence of both male and hermaphrodite light green with pale pink tinged flowers but no female flowers with stamens sometimes expressing polymorphism and fertile gynoecium. Nandan-4 resistant to pest and diseases of Jatropha curcas and more tolerant to environmental stresses especially draught.

The invention involves procedures for creating plants with novel characteristics. In particular, one embodiment of the invention relates to the surprising discovery that the presence of hermaphrodite flowers along with only the male flowers in the interspecific hybrid Nandan-4.

It is produced from interspecific hybridization between two species of Jatropha where Jatropha gossypifolia is the female parent and Jatropha curcas is the pollen parent. The resulting F₁ plant is designated as Nandan-4. The interspecific hybrid showed its unique characters than both the parents, as described in the following hybrid description information.

This invention also directs to the procedures for producing a Jatropha inter or intraspecific hybrid by crossing a first parent Jatropha gossypifolia plant with a second parent Jatropha curcas plant, or vice versa wherein the first or second parent plant is a Jatropha plant from the hybrid Nandan-4. Further, both first and second parent Jatropha plant may be from the hybrid Nandan-4. Therefore, any procedures using the interspecific hybrid Nandan-4 are part of this invention: selfing, backcross, hybrid breeding and crosses to populations. Any plants produced using Jatropha interspecific hybrid Nandan-4 as a parent are within the scope of this invention.

This invention also is directed to procedures for producing Jatropha interspecific hybrid Nandan-4 with a second Jatropha plant and growing the progeny seed, and repeating the crossing and growing steps with the Jatropha interspecific hybrid Nandan-4-derived plant from 0 to 7 times. Thus, any such procedures using the Jatropha interspecific hybrid Nandan-4 are part of this invention: selfing, backcrosses, hybrid production, crosses to populations, and the like. All plants produced using Jatropha interspecific hybrid Nandan-4 as a parent are within the scope of this invention, including plants derived from Jatropha interspecific hybrid Nandan-4.

It should be understood that the parents of interspecific hybrid Nandan-4 can, through routine manipulation of cytoplasmic or other factors, be produced in a male-sterile form. Such embodiments are also contemplated within the scope of the present claims.

Identification of the Marker Molecular Characterization of Nandan-4

### Plant Material

Attempts were made to develop diagnostic molecular markers for the high yielding hybrid Nandan-4. For this purpose, molecular analysis of the hybrid Nandan-4 was carried out with 9 more diverse Jatropha curcas lines that exhibited variability in seed characters (large versus small), maturity pattern (extended flowering versus synchronous maturity), leaf size (small versus medium) and yield (high vs low).

### DNA Extraction

Total genomic DNA was extracted from younger leaves of the hybrid (Nandan-4) following the standard CTAB process with minor modifications (Doyle and Doyle 1987). Five grams of leaves were ground in liquid nitrogen, then homogenized in 20 ml of extraction buffer (2% CTAB, 20 mM EDTA, 2% PVP, 1.4 M NaCl, 100 mM Tris-HCl pH 8.0 and 1% β-mercaptoethanol) and incubated at 65° C. for 1 h. The supernatant was treated with RNase A (100 μg/ml), incubated at 37° C. for 30 min and twice extracted with chloroform: isoamylalcohol (24:1 v/v). The DNA was precipitated with isopropanol and washed twice with 70% ethanol. The pelleted DNA was air dried and resuspended in 500 μl of sterile Millipore water and stored overnight at −20° C.

### RAPD and PCR Analysis

A total of 200 decamer primers from Operon kits—OPB to OPK (Operon technologies, Alameda, USA) were used for DNA amplification according to the process of Williams et al. (1990). The PCR amplification reaction (10 μl) consisted of 2.5 ng of DNA, 1×PCR buffer (10 mM Tris pH 9.0, 50 mM KCl, 1.5 mM MgCl₂), 100 μM of each of the four dNTPs, 0.4 μM of RAPD primer and 0.3 U of Taq DNA polymerase (Bangalore Genei, India). PCR amplifications were performed in an GeneAmp 9700 Thermal Cycler (Eppendorf) with an initial denaturation at 94° C. for 3 min followed by 45 cycles at 94° C. for 45 s, 36° C. for 30 s and 72° C. for 2 min with a final extension at 72° C. for 7 min. The PCR products were separated on 1.5% agarose gel in 1×TAE buffer by electrophoresis at 100 V for 3 h and visualized with ethidium bromide staining under gel documentation system. In general, RAPD markers suffer from a lack of reproducibility, but to check the consistency of the electrophoretic patterns and the polymorphism detected, every PCR reaction was repeated twice. All the PCR amplifications included a negative control (no DNA) to avoid erroneous interpretations.

The 200 tested primers gave robust amplification profiles. The polymorphism detected and polymorphic bands were checked for accession specific bands. Only one marker was found specific to Nandan-4.

Characterization using RAPD and ISSR primers confirms the hybridity. The gel pictures confirm the similarity of the material with J. tanjorensis and also the hybridity between J. curcas and J. gossypifolia. The banding pattern of the material is exactly the same as that of J. tanjorensis. Further, the distinction of the hybrid Nandan-4 has been accomplished through development of a molecular marker specific to the hybrid. The molecular marker's M13 Primer confirming Nandan-4. The inheritance of the markers were validated by checking it on progeny (20) resulting from this promising hybrid

Further reproduction of the hybrid can occur by tissue culture and regeneration. Tissue culture of various tissues of Jatropha species and regeneration of plants there from is well known and widely published. For example, reference may be had to Lin et. al., Plant Physiol Commun 38: 252 (2002); Lu et. al., Environ. Biol. 9: 127 (2003); Sujatha et. al., Plant Cell Tiss. Org. Cult. 44: 135 (1996); and Wei Qin, J. Plant Physiol. Mol. Bio. 30: 475 (2004). Thus, another aspect of this invention is to provide cells which upon growth and differentiation produce Jatropha plants having the physiological and morphological characteristics of Jatropha interspecific hybrid Nandan-4.

As used herein, the term ‘plant’ includes plant cells, plant protoplasts, plant cells of tissue culture from which Jatropha curcas plants can be regenerated, plant calli, plant meristems, and plant cells that are intact in plants or parts of plants, such as pollen, flowers, embryos, ovules, seeds, inflorescence, leaves, stems, pistils, anthers and the like. Thus another aspect of this invention is to provide for cells which upon growth and differentiation produce a cultivar having essentially all of the physiological and morphological characteristics of Nandan-4.

The utility of Jatropha interspecific hybrid Nandan-4 also extends to crosses with other species. Commonly, suitable species will be of the family Euphorbiaceae, and especially of the genera Jatropha

This invention also is directed to procedures for producing a Jatropha inter or intraspecific hybrid by crossing a first parent Jatropha gossypifolia plant with a second parent Jatropha curcas plant, or vice versa wherein the first or second parent plant is a Jatropha plant from the hybrid Nandan-4. Further, both first and second parent Jatropha plant may be from the hybrid Nandan-4. Therefore, any processes using the interspecific hybrid Nandan-4 are part of this invention: selfing, backcross, hybrid breeding and crosses to populations. Any plants produced using Jatropha interspecific hybrid Nandan-4 as a parent are within the scope of this invention: selfing, backcross, hybrid production, crosses to populations and the like. All plants produced using Jatropha interspecific hybrid Nandan-4 as a parent are within the scope of this invention, including those developed from varieties derived from Jatropha interspecific hybrid Nandan-4. Advantageously, the Jatropha interspecific hybrid of the present invention could be used in crosses with other, different, Jatropha species to produce the first filial generation Jatropha hybrid seeds and plants with superior characteristics. Nandan-4 can also be used for transformation where exogenous genes are introduced and expressed by any of a number protocol known to these of skill in the art are intended to be within the scope of this invention.

The following describes breeding procedures that may be used with interspecific hybrid Nandan-4 in the development of further Jatropha plants. One such embodiments is a procedure for developing an Nandan-4-derived progeny Jatropha plant in a breeding program comprising: obtaining the Jatropha plant, or a part thereof, of Jatropha interspecific hybrid plant Nandan-4, utilizing said plant or plant part as a source of breeding material and selecting an Nandan-4 progeny plant with molecular markers in common with Nandan-4 and/or with morphological and/or physiological characteristics selected from the characteristics listed in Table 1. Breeding steps that may be used in the Jatropha breeding program include pedigree breeding, mutation breeding, and recurrent selection. In conjugation with these steps, techniques such as RFLP-enhanced selection, genetic marker enhanced selection like SSR markers and the making of double haploids may be utilized.

One of ordinary skill in the art of plant breeding would know how to evaluate the traits of two plant types to determine if there is no significant difference between the two traits expressed by those plant types. Thus the invention includes Jatropha interspecific hybrid Nandan-4 progeny plants comprising a combination of at least two Nandan-4 traits. Using techniques described herein, molecular markers may be used to identify said progeny plant as a Nandan-4 progeny plant. Mean trait values may be used to determine whether trait difference are significant, and preferably the traits are measured on plants grown under the same environmental conditions. Once such a variety is developed its value is substantial since it is important to advance the germplasm base as a whole in order to maintain or improve traits such as yield, disease resistance, insect pest resistance, and plant performance in extreme environmental conditions.

Progeny of Jatropha interspecific hybrid Nandan-4 may also be characterized through their filial relationship with Nandan-4, as for example, being within a certain number of breeding crosses of Nandan-4. A breeding cross is a cross made to introduce new genetics into the progeny, and is distinguished from a cross, such as a self or a sib cross, made to select among existing genetic alleles. The lower the number of breeding crosses in the pedigree, the closer the relationship between Nandan-4 and its progeny.

### Tables

The following tables present data on the traits and characteristics of Jatropha interspecific hybrid Nandan-4 as compare to its parental lines Jatropha gossypifolia and Jatropha curcas as well as the natural interspecific hybrid Jatropha tanjorensis being Jatropha curcas as female and Jatropha gossypifolia as pollen parent. The results in Table 2 compare the distinctive morphological characters of Nandan-4 with its parents and Jatropha tanjorensis.

TABLE 1
INTERSPECIFIC HYBRID DESCRIPTION INFORMATION
Plant morphological characters

| Plant height | 247 cm |
|---|---|
| Basal girth | 17 cm |
| Canopy diameter, across | 225 cm |
| No. of primary branches | 6 |
| No. of secondary branches | 15 |
| No. of tertiary branches | 30 |

Stem

| | |
|---|---|
| Stem | Medium thick, stout |
| Internodal length | 20 cm |
| Branching pattern | Divergent, Profuse |
| Location of branches | Basal |
| Pigmentation | Sparse |
| Pubescence | Present |

Leaf

| | |
|---|---|
| Size | Medium to large |
| Shape | Palmate, 4-5 lobes |
| Margins | Distantly serrate |
| Color | Light to dark green, no pigmentation |
| Petiole length | 10.6 cm |
| Petiole color | Reddish green |
| Leaf aestivation | Alternate |
| Pubescence | Present |

Inflorescence and flowers

| | |
|---|---|
| Inflorescence type | Compact cymose with co-inflorescence |
| Synchronism in flowering | Present |
| Female to male ratio | 1:5 |
| Flowers | Male and hermaphrodite |
| Flower color | Light green with pale pink tinge |
| Pollen | Polymorphic |
| Gynoecium | Fertile |

Capsule and seed

| | |
|---|---|
| Capsule size | Medium, triloculate with lobes |
| Pedicel length | 1.52 cm |
| Seed size | Medium |
| Seed coat color | Black |
| Shape class | Long |

Disease resistance

| | |
|---|---|
| Powdery mildew | Resistant |
| (Erysiphe euphorbiae) | |

Insect pest resistance

| | |
|---|---|
| Inflorescence & capsule borer | Resistant |
| (Pempelia morosalis) | |
| Leaf miner (Neurobathra | Resistant |
| curcassi Busck.) | |
| Bugs (Scutellera nobilis Fabr.) | Resistant |

TABLE 2

| | J. gossypifolia | Nandan-4 | J. curcas | J. tanjorensis |
|---|---|---|---|---|
| Stem | Medium thick | Medium thick stout | Thick stout stem with | Medium thick stout |
| | stout stem with | stem with sparse | bushy and profuse | stem with sparse |
| | bushy and profuse | branching, more | branching, no | branching, sparse |
| | branching, dense | internodal length, | pigmentation | pigmentation |
| | pigmentation, | sparse pigmentation, | | |
| | pubescence | pubescence present | | |
| Leaves | Leaves alternate, | Leaves alternate, | Alternate, cordate, five | Leaves alternate, |
| | palmately lobed | palmately five lobed, | lobed, light green to dark | palmately five |
| | with 3-5 lobes, | light green to dark | green with no | lobed, light green to |
| | dark green with | green with no | pigmentation on either | dark green with no |
| | dense | pigmentation except | side except on very young | pigmentation except |
| | pigmentation, | on very young leaves, | leaves, long petiole | on very young |
| | margin serrate, | margins distantly | | leaves, margins |
| | petiole with dense | serrate, medium long | | distantly serrate, |
| | pigmentation and | petiole with dense | | long petiole with |
| | glands | reddish green | | dense pigmentation |
| | | pigmentation, | | |
| | | pubescence present | | |
| Inflorescence | Cymose | Cymose | Cymose inflorescence | Cymose |
| | inflorescence with | inflorescence with | with distinct | inflorescence with |
| | coinflorescence | coinflorescence | cofloresecence | coinflorescence |
| Flowers | Monoecious, | Monoecious | Unisexual, monoecious, | Monoecious |
| | unisexual, small | unisexual and | small yellowish green | unisexual and |
| | purple red | hermaphrodite, | flowers with 10 lemon | bisexual, medium, |
| | flowers, 6-8 | medium, sized green | yellow stamens arranged | sized green with |
| | yellow stamens | with pale pink tinged | in a single layer, | pale pink tinged |
| | arranged in two | flowers, 8 yellow | | flowers, 8 yellow |
| | whorls, | stamens arranged in a | | stamens arranged in |
| | | single layer | | a single layer, |
| Carpel | Purple | Creamy | Light greenish | Creamy |
| Pollen | Highly fertile | Highly sterile/fertile | Highly fertile pollen | Highly sterile pollen |
| viability | pollen | pollen | | |
| Fruits | Small deeply | Marginal size Fruit | Large drupaceous fruits | Marginal size Fruit |
| | lobed capsule | | | |
| Seed | Triloculate with | Triloculate with | Triloculate with bigger | Triloculate with |
| | small size | medium size | size | medium size |
| Gynoecium | Fertile | Fertile | Fertile | Sterile |

1. An interspecific hybrid of Jatropha curcas plant designated as Nandan-4 with functional gynoecium produced by crossing Jatropha gossypifolia as female parent with Jatropha curcas as pollen parent.

2. An interspecific Jatropha curcas hybrid (Nandan-4) plant or a part thereof, produced by asexual propagation or tissue culture procedure or growing the seed of the claim 1.

3. Pollen of the hybrid (Nandan-4) plant of claim 2.

4. An ovule of the hybrid (Nandan-4) plant of claim 2

5. A Jatropha curcas hybrid (Nandan-4) plant, or a part thereof, is having all of the physiological and morphological characteristics of the interspecific Jatropha plant of claim 2.

6. A protoplast produced from the hybrid (Nandan-4) plant of claim 1.

7. Tissue culture cells produced from the interspecific Jatropha curcas hybrid (Nandan-4) plant of claim 1, wherein said cells of the tissue culture are produced from a plant part selected from the group consisting of leaves, pollen, embryos, cotyledons, hypocotyls, meristematic cells, roots, root tips, pistils, anthers, flowers, inflorescence, and stems.

8. An inter specific Jatropha curcas hybrid (Nandan-4) plant regenerated from the tissue culture of claim 7 wherein the plant has all the morphological and physiological characteristics of interspecific hybrid Nandan-4.

9. A process of producing the Jatropha interspecific hybrid Nandan-4 by crossing Jatropha gossypifolia as female parent with Jatropha curcas as pollen parent.

10. An interspecific hybrid Nandan-4 plant or part thereof prepared by the process of claim 9.

11. A process of producing an interspecific hybrid of Jatropha plant with the morphological and physiological characteristics of hybrid Nandan-4 according to claim 9, comprising regeneration of a tissue culture.

12. A process of producing an inter specific hybrid of Jatropha plant designated as Nandan-4 according to claim 9 wherein the Nandan-4 exhibits a high yield, resistance to disease and insect pests, improved canopy structure, tolerance to environmental stresses, improved agronomic characteristics and/or higher seed yield.

13. A process of producing an inter specific hybrid of Jatropha plant designated as Nandan-4 according to claim 9, wherein the female parent used in the method comprises the characteristics of producing cymase inflorescence, a highly fertile ovule and a small number of abortive flowers.

14. A process of producing an inter specific hybrid of Jatropha plant designated as Nandan-4 according to claim 9, wherein the male parent used in the method comprises the characteristics of producing a greater number of inflorescences per plant.

15. A process of producing an inter specific hybrid of Jatropha plant according to claim 9, wherein the inter specific hybrid Nandan-4 of Jatropha plant is then crossed with a Jatropha curcas or part thereof, to produce a population of Nandan-4 progeny comprising 20 to 70% of alleles from the Nandan-4 parent.

16. A process of producing an inter specific hybrid of Jatropha plant designated Nandan-4 according to claim 9, wherein the Nandan-4 product comprises the following sequence SEQ. ID. 4 (M1 3 Primer)

17. A process of producing an inter specific hybrid of Jatropha plant designated Nandan-4 according to claim 9, wherein part of the Nandan-4 product is comprising following sequences SEQ. ID. 4 (M1 3 Primer)

18. A process of producing an inter specific hybrid Nandan-4 of Jatropha plant according to claim 9 wherein the Nandan-4 product comprises a sterile male form.

19. An inter specific hybrid Nandan-4 plant or part thereof prepared by the method of claim 9.

20. A process of producing pollen from an inter specific hybrid of Jatropha plant designated as Nandan-4, comprising forming the plant by the method of claim 9 and extracting pollen therefrom.

21. A process of producing an ovule from an inter specific hybrid of Jatropha plant designated as Nandan-4, comprising forming the plant by the method of claim 9 and extracting an ovule therefrom.

22. A process of producing a protoplast from an inter specific hybrid of Jatropha plant designated as Nandan-4, comprising forming the plant by the method of claim 9 and extracting a protoplast therefrom.

23. A process of producing a microbiological product from an inter specific hybrid of Jatropha plant designated as Nandan-4 comprising forming the plant by the method of claim 9 and extracting a microbiological product.

24. A process of producing a microbiological product from an inter specific hybrid of Jatropha plant designated as Nandan-4 comprising forming the plant by the method of claim 9 and extracting a microbiological product selected from either a pollen cell or an ovule cell.

25. A process of producing a microbiological product from an inter specific hybrid of Jatropha plant designated as Nandan-4 comprising forming the plant by the method of claim 9 and extracting a microbiological product comprising one or more of embryos, protoplasts, meristematic cells, callus, leaves, anthers, pistils, root tips, seeds or stems.

26. A process of culturing tissue cells from an inter specific hybrid of Jatropha plant according to claim 9, comprising culturing tissue cells produced from the Jatropha plant, where cells are selected from the group consisting of leaves, pollen, embryos, cotyledons, hypocotyls, meristematic cells, roots, root tips, pistils, anthers, flowers, inflorescence, callus, seeds and stems.

27. Use of the interspecific hybrid Nandan-4 plant or part thereof, according to claim 19 in a process for the production of a biofuel.

28. Use according to claim 27 wherein the biofuel is biodiesel.

29. A biofuel generated by the use according to claim 27.
